(19) United States
(12) Patent Application Publication
(10) Pub. No.: US 2025/0366744 A1
(43) Pub. Date: Dec. 4, 2025

(54) MINIATURE PH SENSOR IN A SUBCUTANEOUS INJECTION NEEDLE FOR BIOFLUID SENSING

(71) Applicant: Southern Methodist University, Dallas, TX (US)

(72) Inventors: Jungchih Chiao, Grand Prairie, TX (US); Khengdauliu Chawang, Dallas, TX (US)

(21) Appl. No.: 19/220,863

(22) Filed: May 28, 2025

Related U.S. Application Data
(60) Provisional application No. 63/653,549, filed on May 30, 2024.

(51) Int. Cl. A61B 5/145; A61B 5/00; A61B 5/1473

(52) U.S. Cl. CPC A61B 5/14539; A61B 5/14735; A61B 5/6848; A61B 2562/125

(57) ABSTRACT

Provided herein are systems and methods related to a pH-sensing electrode including a substrate; a working-material layer disposed on a first surface of the substrate; and a reference-material layer disposed on a second surface of the substrate, wherein the second surface is opposite the first surface; wherein the pH-sensing electrode is configured to be inserted into a needle for placement in or into a tissue, or completely or partially inside a blood vessel.

### CROSS-REFERENCE TO RELATED APPLICATIONS

This application claims priority to U.S. Provisional Application Ser. No. 63/653,549, filed May 30, 2024, the entire contents of which is incorporated herein by reference.

### STATEMENT OF FEDERALLY FUNDED RESEARCH

Not applicable.

### TECHNICAL FIELD OF THE INVENTION

The present invention relates in general to pH sensing, and more particularly, to the use of an electrode to sense pH.

### BACKGROUND OF THE INVENTION

Without limiting the scope of the invention, its background is described in connection with sensing pH in human blood serum.

The pH value in bodily fluids is a crucial diagnostic marker. Conventional glass-rod pH sensors display reliability in aqueous solutions, but their bulky design hinders miniaturization, and the pH-sensitive glass membrane makes them prone to inaccuracies in viscous solutions due to elevated junction potentials.

The pH value indicates H⁺ activity in tissues, which is crucial for health assessment [1]. pH change in body fluid shows metabolic states and is frequently monitored for medical diagnosis. For instance, tumor cells proliferate as the extracellular pH changes from normal pH 7.3 to abnormal pH 6.8 [2] leading to vital organ failure due to an acidic environment [3]. Reports suggest skin pH changes from a mean acidic pH 4.7 to an alkaline pH 9 in chronic wounds [4]. Inflammatory responses such as sepsis require early diagnosis for timely treatment [5]. Therefore, pH sensors are crucial for treatment regulation and early diagnosis [6]. Traditional pH-sensitive glass membranes offer stable pH responses in an aqueous solution, but in viscous fluids like blood, protein adsorption causes inaccuracies [7], [8]. Salt bridges can mitigate this effect and prevent contamination, but they are bulky. Recent advances in wearable integrate salt bridges on flexible substrates, but only for skin applications [9], [10]. This work proposes a pH sensor eliminating the need for salt bridges and enabling miniaturization for needle insertion to conduct fluid pH monitoring in tissues.

### SUMMARY OF THE INVENTION

As embodied and broadly described herein, an aspect of the present disclosure relates to a pH-sensing electrode including a substrate; a working-material layer disposed on a first surface of the substrate; and a reference-material layer disposed on a second surface of the substrate, wherein the second surface is opposite the first surface; wherein the pH-sensing electrode is sized for placement in or into a tissue, or completely or partially inside a blood vessel. In one aspect, the substrate comprises at least one of: polyimide, ethylene-tetrafluoroethylene (ETFE), a fluorinated polymer, polyester, polyamide, polyvinylidene fluoride (PVDF), polycarbonate, ETFE/PVDF mixtures, polypropylene, aromatic polyethers, polybenzoxazoles, polyetheretherketones, polybenzimidazoles, glass, epoxy resin, bismaleimide, cyanate ester, vinyl resin, polyethersulfone, polyurethane, gold bonding wire, platinum wire, iridium wire, or combinations thereof. In another aspect, the working-material layer comprises at least one of: iridium oxide, ruthenium oxide, titanium dioxide, tin oxide, tantalum oxide, tungsten oxide, erbium oxide, cerium oxide, or zinc oxide. In another aspect, the working-material layer is deposited on the substrate by at least one of: a sol-gel method, electroplating, electrodeposition, physical vapor deposition, or metal-organic chemical vapor deposition. In another aspect, the reference-material layer comprises at least one of: silver/silver chloride, mercuric oxide, or manganese oxide. In another aspect, the reference-material layer is deposited on the substrate by at least one of: silver/silver chloride ink deposition, electroplating using silver nanoparticles, or electrodeposition using silver nanoparticles. In another aspect, the pH-sensing electrode is inserted into a needle. In another aspect, the one or more pH-sensing electrodes are adapted for subcutaneous, percutaneous, intracutaneous, subdermal, intraperitoneal, intramuscular, intraarticular, periarticular, intraosseous, intraocular, intracardiac, intracavernous, intradetrusor insertion, or insertion into a vein or artery.

As embodied and broadly described herein, an aspect of the present disclosure relates to a pH-sensing array including a plurality of pH-sensing electrodes, each pH-sensing electrode including a substrate; a working-material layer disposed on a first surface of the substrate; and a reference-material layer disposed on a second surface to the substrate, wherein the second surface is opposite the first surface; wherein the pH-sensing electrode is configured for placement in or into a tissue, or completely or partially inside a blood vessel. In one aspect, the plurality of pH-sensing electrodes are adapted for subcutaneous, percutaneous, or intracutaneous insertion. In another aspect, the substrate comprises at least one of: polyimide, ethylene-tetrafluoroethylene (ETFE), a fluorinated polymer, polyester, polyamide, polyvinylidene fluoride (PVDF), polycarbonate, ETFE/PVDF mixtures, polypropylene, aromatic polyethers, polybenzoxazoles, polyetheretherketones, polybenzimidazoles, glass, epoxy resin, bismaleimide, cyanate ester, vinyl resin, polyethersulfone, polyurethane, gold bonding wire, platinum wire, or iridium wire, or combinations thereof. In another aspect, the working-material layer comprises at least one of: iridium oxide, ruthenium oxide, titanium dioxide, tin oxide, tantalum oxide, tungsten oxide, erbium oxide, cerium oxide, or zinc oxide. In another aspect, the working-material layer is deposited on the substrate by at least one of: a sol-gel method, electroplating, electrodeposition, physical vapor deposition, or metal-organic chemical vapor deposition. In another aspect, the reference-material layer comprises at least one of: silver/silver chloride, mercuric oxide, or manganese oxide. In another aspect, the reference-material layer is deposited on the substrate by at least one of: silver/silver chloride ink deposition, electroplating using silver nanoparticles, or electrodeposition using silver nanoparticles. In another aspect, the plurality of pH-sensing electrodes is/are adapted for subcutaneous, percutaneous, intracutaneous, subdermal, intraperitoneal, intramuscular, intraarticular, periarticular, intraosseous, intraocular, intracardiac, intracavernous, intradetrusor insertion, or insertion into a vein or artery.

As embodied and broadly described herein, an aspect of the present disclosure relates to a method of making a pH-sensing electrode including providing a substrate; disposing a working-material layer on a first surface of the substrate; and disposing a reference-material layer on a second surface of the substrate, wherein the second surface is opposite the first surface; wherein the pH-sensing electrode is sized for placement in or into a tissue, or completely or partially inside a blood vessel. In one aspect, the substrate comprises at least one of: polyimide, ethylene-tetrafluoroethylene (ETFE), a fluorinated polymer, polyester, polyamide, polyvinylidene fluoride (PVDF), polycarbonate, ETFE/PVDF mixtures, polypropylene, aromatic polyethers, polybenzoxazoles, polyetheretherketones, polybenzimidazoles, glass, epoxy resin, bismaleimide, cyanate ester, vinyl resin, polyethersulfone, polyurethane, gold bonding wire, platinum wire, iridium wire, or combinations thereof. In another aspect, the working-material layer comprises at least one of: iridium oxide, ruthenium oxide, titanium dioxide, tin oxide, tantalum oxide, tungsten oxide, erbium oxide, cerium oxide, or zinc oxide. In another aspect, the working-material layer is deposited on the substrate by at least one of: a sol-gel method, electroplating, electrodeposition, physical vapor deposition, or metal-organic chemical vapor deposition. In another aspect, the reference-material layer comprises at least one of: silver/silver chloride, mercuric oxide, or manganese oxide. In another aspect, the reference-material layer is deposited on the substrate by at least one of: silver/silver chloride ink deposition, electroplating using silver nanoparticles, or electrodeposition using silver nanoparticles. In another aspect, the one or more pH-sensing electrodes is adapted for insertion into a needle. In another aspect, the one or more pH-sensing electrodes are adapted for subcutaneous, percutaneous, intracutaneous, subdermal, intraperitoneal, i intramuscular, intraarticular, periarticular, intraosseous, intraocular, intracardiac, intracavernous, intradetrusor insertion, or insertion into a vein or artery.

As embodied and broadly described herein, an aspect of the present disclosure relates to a sensing electrode array including a substrate; a working-material layer disposed on a first surface of the substrate and etched to provide two or more electrodes; and a reference-material layer disposed on a second surface of the substrate, wherein the second surface is opposite the first surface; wherein the sensing electrode is sized for placement in or into a tissue, or completely or partially inside a blood vessel; and wherein the sensing electrode is shaped to form part or all of a cylinder and sized to be inserted into a needle. In one aspect, the substrate comprises at least one of: polyimide, ethylene-tetrafluoroethylene (ETFE), a fluorinated polymer, polyester, polyamide, polyvinylidene fluoride (PVDF), polycarbonate, ETFE/PVDF mixtures, polypropylene, aromatic polyethers, polybenzoxazoles, polyetheretherketones, polybenzimidazoles, glass, epoxy resin, bismaleimide, cyanate ester, vinyl resin, polyethersulfone, polyurethane, gold bonding wire, platinum wire, iridium wire, or combinations thereof. In another aspect, the working-material layer comprises at least one of: iridium oxide, ruthenium oxide, titanium dioxide, tin oxide, tantalum oxide, tungsten oxide, erbium oxide, cerium oxide, or zinc oxide. In another aspect, the working-material layer is deposited on the substrate by at least one of: a sol-gel method, electroplating, electrodeposition, physical vapor deposition, or metal-organic chemical vapor deposition. In another aspect, the reference-material layer comprises at least one of: silver/silver chloride, mercuric oxide, or manganese oxide. In another aspect, the reference-material layer is deposited on the substrate by at least one of: silver/silver chloride ink deposition, electroplating using silver nanoparticles, or electrodeposition using silver nanoparticles. In another aspect, the one or more pH-sensing electrodes are adapted for subcutaneous, percutaneous, intracutaneous, subdermal, intraperitoneal, intramuscular, intraarticular, periarticular, intraosseous, intraocular, intracardiac, intracavernous, intradetrusor insertion, or insertion into a vein or artery.

As embodied and broadly described herein, an aspect of the present disclosure relates to a method of making a sensing electrode array including providing a substrate; disposing a working-material layer on a first surface of the substrate; etching the working-material layer to provide two or more electrodes; and disposing a reference-material layer on a second surface of the substrate, wherein the second surface is opposite the first surface; wherein the sensing electrode array is sized for placement in or into a tissue, or completely or partially inside a blood vessel, and wherein the sensing electrode array is shaped to form part or all of a cylinder and sized to be inserted into a needle. In one aspect, the substrate comprises at least one of: polyimide, ethylene-tetrafluoroethylene (ETFE), a fluorinated polymer, polyester, polyamide, polyvinylidene fluoride (PVDF), polycarbonate, ETFE/PVDF mixtures, polypropylene, aromatic polyethers, polybenzoxazoles, polyetheretherketones, polybenzimidazoles, glass, epoxy resin, bismaleimide, cyanate ester, vinyl resin, polyethersulfone, polyurethane, gold bonding wire, platinum wire, iridium wire, or combinations thereof. In another aspect, the working-material layer comprises at least one of: iridium oxide, ruthenium oxide, titanium dioxide, tin oxide, tantalum oxide, tungsten oxide, or zinc oxide. In another aspect, the working-material layer is deposited on the substrate by at least one of: a sol-gel method, electroplating, electrodeposition, physical vapor deposition, or metal-organic chemical vapor deposition. In another aspect, the reference-material layer comprises at least one of: silver/silver chloride, mercuric oxide, or manganese oxide. In another aspect, the reference-material layer is deposited on the substrate by at least one of: silver/silver chloride ink deposition, electroplating using silver nanoparticles, or electrodeposition using silver nanoparticles. In another aspect, the pH-sensing electrode is adapted for insertion into a needle. In another aspect, the one or more pH-sensing electrodes are adapted for subcutaneous, percutaneous, intracutaneous, subdermal, intraperitoneal, intramuscular, intraarticular, periarticular, intraosseous, intraocular, intracardiac, intracavernous, intradetrusor insertion, or insertion into a vein or artery.

### BRIEF DESCRIPTION OF THE DRAWINGS

For a more complete understanding of the features and advantages of the present invention, reference is now made to the detailed description of the invention along with the accompanying figures, in which:

FIG. 1A shows an IrCl₃ solution coated on flexible polyimide by roll-to-roll (R2R) process. FIG. 1B shows the coated polyimide heated for iridium oxide (IrOₓ) development.

FIG. 1C shows Ag/AgCl ink printed on the other side of polyimide. FIG. 1D shows a photo of the electrode. FIG. 1E shows a cross-sectional view of the electrode. FIG. 1F shows a miniature back-to-back (b2b) electrode design. FIG. 1G shows side, top, and bottom views of the electrode. FIG. 1H shows the electrode within a needle or catheter.

FIG. 2A shows potential responses at pH 4, 7, and 10 for a single b2b electrode and single-sided electrodes of IrOₓ vs AgCl that are spaced with a distance from 2 to 80 mm.

FIG. 2B shows the Nernstian response, showing back-to-back fits linearly into the calibration curve. All electrode sizes were 2× 10 mm for consistent comparison.

FIG. 3A shows the OCP measurement of a miniature b2b electrode from pH 9 to 6 with 0.5 pH steps. FIG. 3B shows the sensitivity from OCP measurements.

FIGS. 4A and 4B show electrochemical measurements of a miniature b2b electrode during OCP from pH 7 to 8 with 0.1 pH steps (FIG. 4A); and CV in PBS 1X pH 7.5 with a scan range of −0.8 to 1 V and a scan rate of 10 mV/s (FIG. 4B).

FIGS. 5A and 5B the results of test the miniature b2b electrodes at 25° C. and 37° C. (FIG. 5A); and the corresponding sensitivities (FIG. 5B).

FIG. 6A shows a photo of a 20-gauge stainless needle. FIG. 6B shows a miniature b2b electrode inside a hollow needle. FIG. 6C shows a blood vessel phantom and an illustration of the needle insertion.

FIG. 7 shows the potential drift (V′) for a miniature b2b electrode inserted inside the phantom with PBS and human serum.

FIG. 8 shows the repeatability of miniature b2b electrodes tested in human serum in a Clarke Error Grid.

FIG. 9 shows a back-to-back sensing electrode inside a hollow needle that is inserted into tissue.

FIG. 10A shows a cross-section of a back-to-back sensing electrode, and FIG. 10B shows a perspective view of a back-to-back sensing electrode.

FIG. 11A shows a back-to-back sensing electrode inside a needle. FIG. 11B shows the back-to-back sensing electrode after removal of the needle, leaving the electrode inside tissues and completely or partially inside a blood vessel. FIG. 11C shows the back-to-back sensing electrode with a connection made to sensing equipment.

FIG. 12A shows the use of a needle to insert a back-to-back sensing electrode into a blood vessel through an intravenous catheter. FIG. 12B shows the electrode inside the catheter after removal of the needle, and the electrode, which remains partially or completely in the blood vessel, after removal of the catheter. FIG. 12C shows an alternative, leaving the electrode inside the catheter.

FIG. 13A shows a radio-frequency identification (RFID) implementation of the back-to-back sensing electrode which operates without a battery. FIG. 13B shows the signals received from the electrode.

FIG. 14A shows the use of a back-to-back sensing electrode to detect or monitor a tumor. The tumor pH values are different from healthy tissues. Strategically placing the pH sensor can monitor the size of the tumor under therapy. FIG. 14B shows the use of the electrode to detect a suture leak. After surgery, the pH sensor outside the GI tract detects pH changes to monitor potential leak from the suture site. FIG. 14C shows the use of the electrode to detect pH in tissue such as muscle tissue to detect or monitor pH changes due to bacteria growth or lactic acid accumulation.

FIG. 15 shows a method embodiment of the present invention.

FIGS. 16A-H shows aspects of one embodiment of the electrode of the present invention. FIG. 16A shows a metal layer coating on a flexible polyimide substrate. FIG. 16B shows metal patterning by a photolithography process. FIG. 16C shows conductive strips. FIG. 16D shows IrOₓ coating by a sol-gel process. FIG. 16E shows Ag/AgCl coating by screen printing. FIG. 16F shows a deformed IrOₓ and Ag/AgCl electrodes shaped into a cylinder. FIG. 16G and FIG. 16H show the electrodes on the deformable substrate conformed inside a needle or catheter and the wires for signal transduction.

FIGS. 17A-H show aspects of another embodiment of biomarker-sensitive and electrodes utilizing the IrOₓ and Ag/AgCl as working and reference electrodes inside a needle or catheter. FIG. 17A shows a metal layer coating on a flexible polyimide substrate. FIG. 17B shows metal patterning by a photolithography process. FIG. 17C shows conductive strips. FIG. 17D shows IrOₓ coating by a sol-gel process before Ag/AgCl coating by screen printing. FIG. 17E shows a patterned coating of photoresist to open the electrodes for liquid contacts and protect the connection wires from short-circuit. FIG. 17F shows a deformed multiplexed electrode shaped into a cylinder. FIG. 17G shows the electrodes on the deformed substrate conformed inside a needle or catheter and the wires for signal transduction. FIG. 17H shows side and top views of the electrode, a cross-section of the electrodes on the deformable substrate shaped into a cylinder, and the electrodes on the deformed substrate conformed inside a needle or catheter and the wires for signal transduction.

FIG. 18 shows another method embodiment of the present invention.

### DETAILED DESCRIPTION OF THE INVENTION

Illustrative embodiments of the system of the present application are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

The pH value in bodily fluids is a crucial diagnostic marker. Conventional glass-rod pH sensors display reliability in aqueous solutions, but their bulky design hinders miniaturization, and the pH-sensitive glass membrane makes them prone to inaccuracies in viscous solutions due to elevated junction potentials. To overcome the issues presented by conventional glass-rod pH sensors, described above, the present invention introduces a new pH sensor design and fabrication that not only enables miniaturization and reliability in aqueous and viscous solutions but also facilitates insertion into a needle for in vivo monitoring. Utilizing a printing technique for the application of iridium oxide and silver/silver chloride coating on a flexible polyimide substrate offers cost-effectiveness and production scalability. The sensor then is tailored with a sharp blade to a narrow strip that fits into a needle such as a 20-, 28-, or 31-gauge needle. The electrodes produced through this method demonstrate promise through electrochemical measurements in buffer solutions, cyclic voltammetry analysis, and real human serum tests.

Biofluid sensing requires miniature pH electrodes to exhibit biocompatibility, sensitivity in the physiological pH range, and quick response. Although commonly used pH-sensitive materials like hydrogen ionophores (HI) and polyaniline (PANI) demonstrate excellent pH characteristics, they raise concerns regarding biocompatibility [11]. Microelectrodes coated with HIs such as tridodecy-lamine and 4-nonadecylpyridine are suggested to be biotoxic during direct skin contact [12]. PANI with low-molecular weight benzidine as a byproduct has been studied to be cytotoxic and carcinogenic [13]. Therefore, for a detection platform, these materials are suggested to avoid open skin or tissue contact [14].

Most pH-sensitive metal oxides coated on flexible polyimide substrates show negligible toxicity and allow size scalability [15]. Among these, iridium oxide (IrOₓ) stands out for its biocompatibility and inert nature, extensively researched in physiological and microscopic settings [16]. Reports demonstrate IrOₓ possesses desirable properties such as high charge injection, making it suitable for neural implants without damaging surrounding tissue [17]. Size scalability and substrate flexibility enable continuous blood pH monitoring from veins with a minimal blood sample volume as demonstrated in animal experiments [18]. The Nernstian relationship between potential and pH over a wide pH range [19], biocompatibility [20], quick response in aqueous and non-aqueous solutions [21], and selectivity features make IrOₓ an attractive choice for physiological measurement purposes. Common IrOₓ deposition includes thermal, radio-frequency (RF) magnetron sputtering, and sol-gel processes. While thermal and sputtering methods necessitate vacuum systems, elevating processing costs, sputtered IrOₓ films (SIROF) display robust adhesion and have been investigated for neural stimulation [23]. However, the coating process is not economical due to material waste from high-purity target discs. To reduce material waste, recent efforts have been made to create uniform flux and prevent preferential material usage from circular areas [24]. The additional steps to reclaim material waste and vacuum units make thermal and SIROF expensive. Sol-gel deposition does not require vacuum systems and waste is minimized since deposition is equal to consumption during film growth. Sol-gel provides good adhesion and the film thickness can be increased by multiple dipping [26]. Our group demonstrated a sol-gel IrOₓ film coated on polyimide with a thermal tolerance of 400° C. and the oxide layer is formed at 325° C. [27]-[29]. Previous design was a typical potentiometric pH sensor with two electrodes, one operating as the working electrode and the other as the reference electrode [27]-[29]. This paper reports a new design with IrOₓ as a working electrode and Ag/AgCl as a reference electrode, both printed back-to-back on a single polyimide film. For applications, the electrode was miniaturized to 0.5×1 mm² to fit inside a 20-gauge needle for detecting a small fluid volume inside tissues. However, needles of other gauges such as 12, 14, 16, 18, 22 or higher can be used. The needle can be made from any material, such as, metal, plastic, glass, ceramic, polymeric, or combinations thereof. The needle(s) can able be coated.

Substrates for use with the present invention include but are not limited to at least one of: polyimide, ethylene-tetrafluoroethylene (ETFE), a fluorinated polymer, polyester, polyamide, polyvinylidene fluoride (PVDF), polycarbonate, ETFE/PVDF mixtures, polypropylene, aromatic polyethers, polybenzoxazoles, polyetheretherketones, polybenzimidazoles, glass, epoxy resin, bismaleimide, cyanate ester, vinyl resin, polyethersulfone, polyurethane, gold bonding wire, platinum wire, iridium wire, or combinations thereof. The working-material layer for use with the present invention includes but is not limited to at least one of: iridium oxide, ruthenium oxide, titanium dioxide, tin oxide, tantalum oxide, tungsten oxide, erbium oxide, cerium oxide, or zinc oxide. The working-material layer can be deposited on the substrate that includes but is not limited to at least one of: a sol-gel method, electroplating, electrodeposition, physical vapor deposition, or metal-organic chemical vapor deposition. The reference-material layer includes but is not limited to at least one of: silver/silver chloride, mercuric oxide, or manganese oxide. The reference-material layer is deposited on the substrate by one or more methods that include but are not limited to by at least one of: silver/silver chloride ink deposition (including, e.g., roll-to-roll printing, roll printing, ink jet printing, aerosol printing, and screen printing), electroplating using silver nanoparticles, or electrodeposition using silver nanoparticles.

Initial measurements such as open-circuit potential (OCP) and cyclic voltammetry (CV) in buffer showed promising results with 0.5 pH and 0.1 pH changes. Detecting small changes is crucial to identify metabolic state since blood pH range is 7.35-7.45. Miniature back-to-back electrodes were further probed into a phantom for monitoring human serum with 0.1 pH changes. Clarke Error Grid analysis demonstrated the repeatability feature of the sensing electrode.

### Electrode Fabrication and Materials

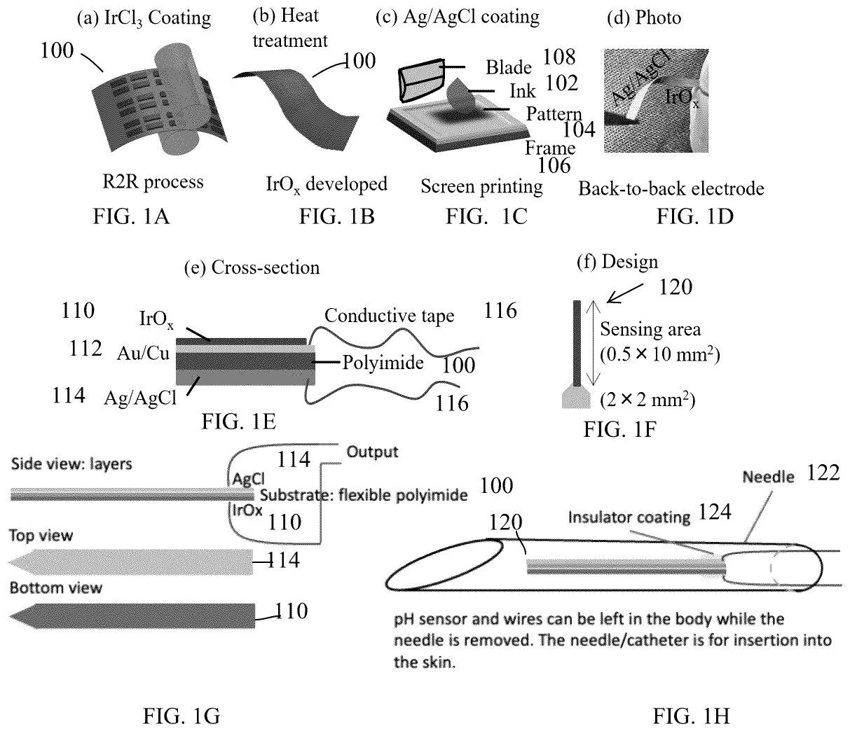

The fabrication procedures include one of several non-limiting options that can be used to fabricate all of the electrode or electrode-array configurations disclosed herein. One of these fabrication options includes the following steps. The IrOₓ is deposited and oxidized on a complete piece of the substrate. Then the substrate is screen-printed with Ag/AgCl on the other side. After the Ag/AgCl is dried, a sharp blade or other cutting tool such as a laser tailors the shape of electrode shaft. The cutting of the shaft can also be accomplished by laser cutting. In one specific fabrication of the electrode of the present invention using this option, flexible polyimide (Sheldahl, USA) was coated with copper (Cu) and gold (Au) layers (18 nm and 90 nm thick, respectively). Anhydrous iridium (Sigma, USA), ethyl alcohol (Supelco, USA), and 80% acetic acid (Labchem, USA) were used for the sol-gel solution. The iridium chloride (IrCl₃) solution was coated on the polymeric substrate 100 using a roll-to-roll (R2R) process, as shown in FIG. 1A [29]. The substrate 100 was heated at a rate of 1° C./min, held at 325° C. for 4 hours to form iridium oxide (IrOₓ), and then cooled at −1° C./min, as shown in FIG. 1B. The Ag/AgCl paste was applied to the backside of the substrate 100 (not shown in FIG. 1C because obscured by the ink 102, the pattern 104, and the frame 106) using screen printing, as shown in FIG. 1C. (The substrate 100 is not shown in FIG. 1C because obscured by the ink 102, the pattern 104, and the frame 106, which are used in the screen-printing process.) The electrode is then tailored with a sharp blade 108 or other cutting tool such as a laser to a narrow strip that fits into a needle such as a 20-, 28-, or 31-gauge needle, as shown in FIG. 1C. An electrode photo is shown in FIG. 1D and a cross-sectional view is shown in FIG. 1E, including an IrOₓ layer 110, a gold Au/Cu layer 112, the substrate 100, an Ag/AgCl layer 114, and conductive tapes 116. The miniature back-to-back (b2b) electrode 120, designed for probing in a phantom, is shown in FIG. 1F with a sensing area of 0.5×1 mm², and an electrical connection pad of 2×2 mm². FIG. 1G shows a side view, including the Ag/AgCl layer 114, the substrate 100, and the IrOₓ layer; a top view including the Ag/AgCl layer 114; and a bottom view including IrOₓ layer. FIG. 1H shows the electrode within a needle, including the electrode 120, a needle 122, and an insulator coating 124.

Custom buffers were made by mixing commercial buffers with 0.05-M sodium chloride salt (NaCl) (Fisher, USA) to enhance solution conductivity. For biological relevance, miniature electrodes were tested in phosphate-buffered saline (1X PBS) (Fisher, USA) with a 0.137-M NaCl concentration [30]. pH levels of PBS were adjusted with hydrochloric acid (HCl) (LabChem, USA) and sodium hydroxide (NaOH) (Sigma-Aldrich, USA). Buffer solution temperatures were monitored with a digital thermometer (Elitech, USA). A commercial pH sensor (Apera, USA) measured human serum pH (Sigma-Aldrich, USA). A phantom with a silicone tube mimicked blood vessels for the body fluid experiments. Electrochemical techniques, including open-circuit potential (OCP) and cyclic voltammetry (CV), were conducted using a potentiostat (CH Instrument, USA). The electrical interface and data recording were established using a data acquisition card (National Instruments, USA), controlled by a LabView program, with a sampling rate of 1 S/s.

### Results and Discussion

### Back-to-Back Design Compared to Two-Electrode Design.

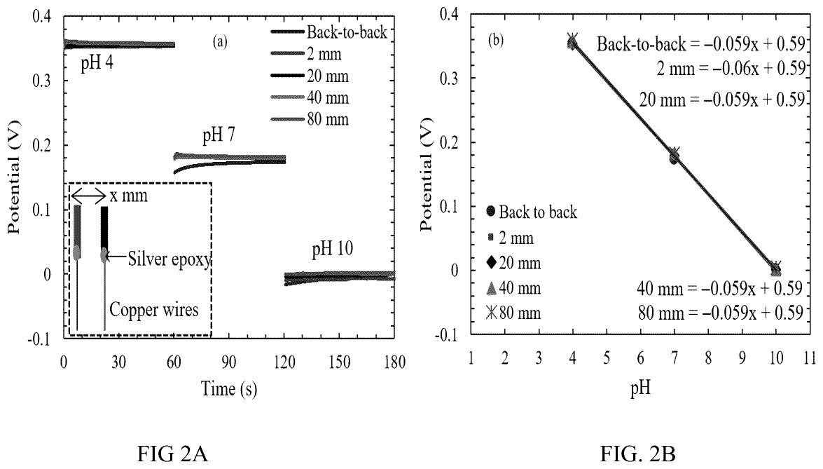

The back-to-back (b2b) printed electrode 120 was tested in custom buffers and compared to a two-electrode design from previous studies [29]. The inset in FIG. 2A illustrates the two-electrode design configuration where IrOₓ (green) and Ag/AgCl electrodes (black) are separated by “x” mm distance ranging from 2 to 80 mm. All electrode sizes were 2×10 mm² and tested in custom-made buffers pH 4, 7, and 10. The electrodes were tested for 60 s in all pH levels. FIG. 2A shows that the potential responses for the back-to-back electrode 120 are consistent with those by two-electrode designs. FIG. 2B demonstrates that regardless of electrode spacing, the Nernstian sensitivities of −59 mV/pH are maintained. This shows printing IrOₓ and Ag/AgCl back-to-back (b2b) does not change the Nernstian responses dramatically.

### Miniature Back-to-Back Electrode in PBS.

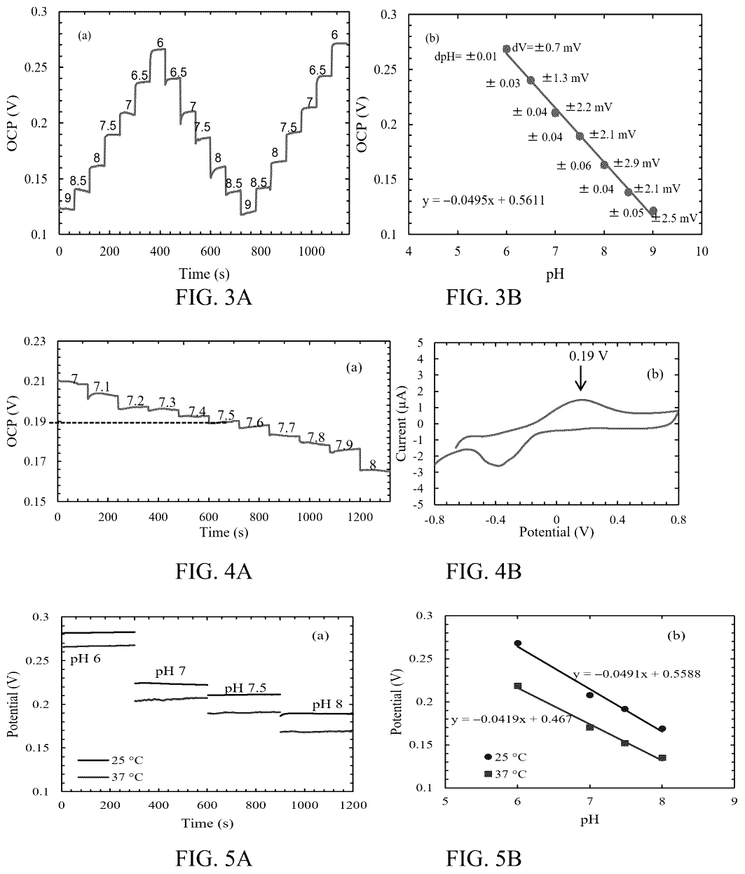

The back-to-back (b2b) electrode 120 was further miniaturized to 0.5×1 mm², as shown in FIGS. 1A-H. For biofluid applications, this electrode 120 was tested in 1×PBS from pH 6 to 9 for biological relevance. All 1×PBS solutions had 0.137-M NaCl salt concentration close to human body fluid [30]. FIG. 3A shows the open-circuit potential (OCP) measurements for 60 s at each pH solutions from pH 6 to 9. Electrodes 120 (not shown) were cleaned in deionized (DI) water between every test. The output potential changed with every 0.5 pH step repeatability. The electrode 120 (not shown) showed a sensitivity of −49.5 mV/pH in FIG. 3B. The reduced surface area of 0.5×1 mm² could account for the lower sensitivity, compared to −59 mV/pH of 2×10 mm² electrodes. Liao et al. demonstrated sensitivity of metal oxide is influenced by the number of hydroxyl groups per unit surface area [31].

The miniature b2b electrode 120 (not shown) demonstrated acceptable hysteresis (dV) and their corresponding pH variation (dpH), indicated by the error bars in FIG. 3(B). Hysteresis (dV) was previously defined as the standard error of the potential difference at the same pH level [29]. The “±” sign in the dV indicates an average variation between the maxima and minima potentials. Hysteresis was in the range of ±(0.7-2.9) mV with a corresponding pH variation (dpH) range of ±(0.01-0.06) in various aqueous buffer solutions.

FIG. 4A shows the miniature b2b electrode 120 (not shown) tested continuously from pH 7 to 8 without DI cleaning. Each test continued for 120 s and the output potential changed with every 0.1 pH step change. The dotted line at pH 7.5 showed a stable OCP output of 0.19 V comparable to the anodic peak in FIG. 4B during the CV measurement. Similar anodic and cathodic enhanced areas illustrate reversible electrochemical mechanisms. For CV, the miniature 0.5×1 mm² IrOₓ was tested against a commercial glass-rod Ag/AgCl electrode and a platinum foil used as reference and counter electrodes, respectively. The CV study using a commercial Ag/AgCl electrode corroborates the stable and repeatable performance of a miniature b2b electrode 120.

### Temperature Effects.

FIG. 5A shows miniature b2b electrodes 120 (not shown) were first tested from pH 6 to 8 at the room temperature of 25° C. and then switched to 37° C. Tests were performed in a 30 mL beaker and a digital thermometer tracked temperature rise to 37° C. The electrodes were cleaned in DI water between tests to remove surface residues. The output potential was recorded for 5 minutes at pH 6, 7, 7.5, and 8. The blue and red lines indicate tests at 25 and 37° C., respectively. The decreased output potentials at 37° C. are consistent with previous reports [29]. FIG. 5B shows the comparable sensitivities of −49.1 mV/pH and −41.9 mV/pH at 25 and 37° C. The sensitivities were linear at both temperatures.

### Miniature b2b Electrode Tested in Human Serum.

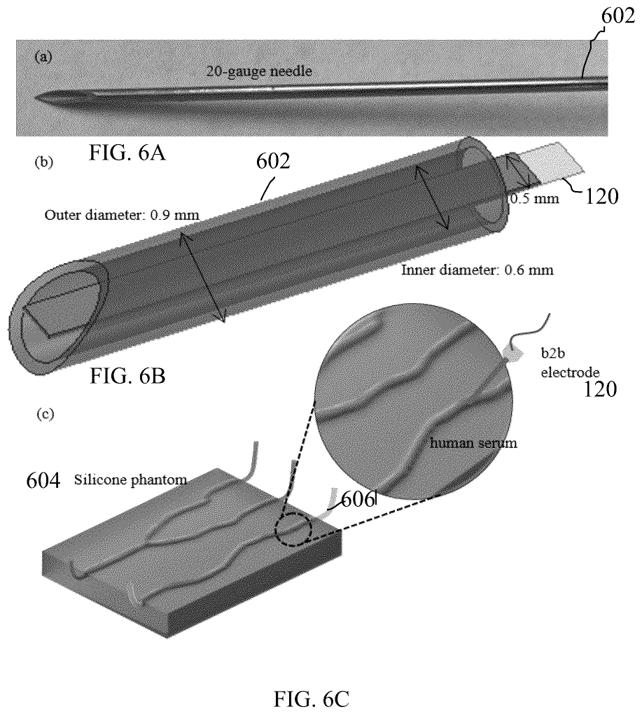

FIG. 6A shows a 20-gauge hollow needle 602 with a size safe for biopsy sampling in clinical practices and FIG. 6B shows the inner and outer diameters of 0.6 mm and 0.9 mm of the needle 602, respectively, for the placement of the miniature b2b electrode 120 with a width of 0.5 mm inserted inside the needle. The figure also demonstrates the miniature b2b electrode 120 size of 0.5 mm is inserted inside the needle. For the fluid test, FIG. 6C shows a silicone phantom 604 with an artificial vessel tube 606 filled with 1 mL of real human serum. The needle was used as a guide to probe the miniature b2b electrode 120 inside the silicone tube 606 filled with human serum. The phantom tube 606 diameter of 5 mm resembles the average human vein diameter typically of 7-15 mm [33]. The calculated amount of liquid in contact with the pH probe 120 was 10 μL.

Four solutions were used, PBS solution with pH 7.5 and human serums with pH levels 7.68, 7.9, and 8. A commercial hand-held pH meter measured the as-received human serum as pH 8. To adjust pH levels, directly mixing concentrated 16.456-M HCl with human serum produced an unreliable and noisy output potentials from the meter. Mixing PBS solutions and human serum at a fixed ratio produced reasonable results. For example, serum pH 8 and PBS pH 7.5 solution at a ratio of 1:1 produced pH 7.9 while serum pH 8 and PBS pH 6 solution in a ratio of 2:1 produced pH 7.68.

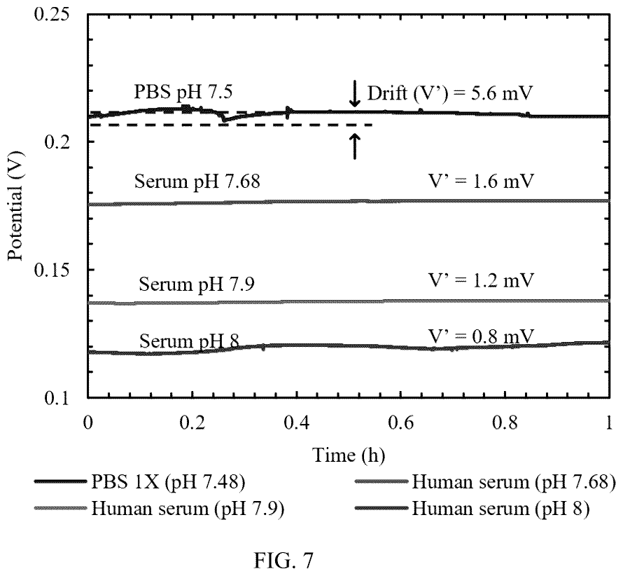

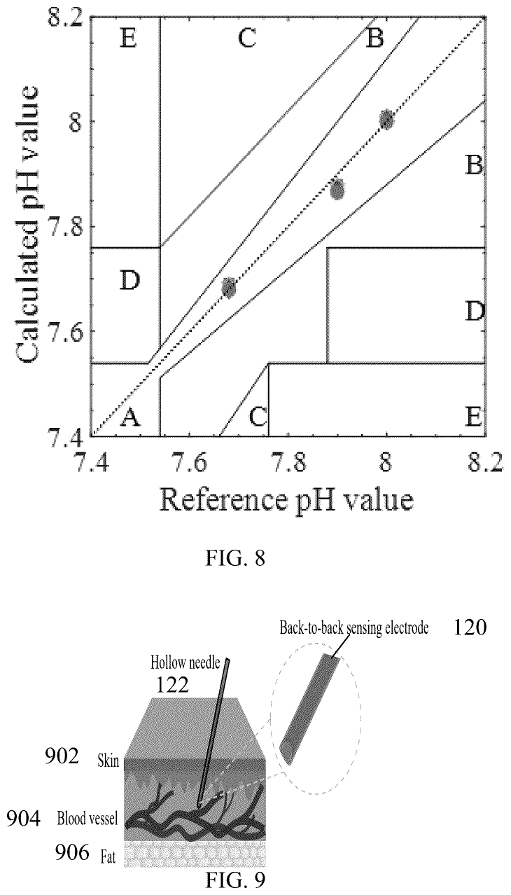

Four newly-made miniature b2b electrodes 120 were tested in the four solutions inside the phantom 604 as shown in FIG. 7 (electrode 120 and phantom 604 not shown). The results are shown in FIG. 7. The electrodes 120 showed low potential drift (V′) when tested for 1 h, indicated by the dotted line at pH 7.5. Potential drift was previously defined as the difference between the initial potential shoot and settled potential [29]. The V′ at pH 7.5, 7.68, 7.9, and 8 were 5.6, 1.6, 1.2, and 0.8 mV, respectively. The electrode 120 (not shown) showed a stable and distinct response to a 0.1 pH step change in human serum. To demonstrate repeatability, three new electrodes 120 (not shown) designed for pH levels of 7.68, 7.9, and 8 underwent ten tests each in serum. Two calibration methods were employed using data from PBS and serum. The first calibration used the output potentials of PBS at pH 7.5 and serum pH 8 to establish the potential-pH slope. The maximum pH variations from such a calibration were 0.01, 0.01, and 0.05 at pH levels 7.68, 7.9, and 8, respectively. The second calibration method utilized output potentials at pH levels of 7.68, 7.9, and 8 of serum, resulting in reduced variations. After the calibration slope was established, among the subsequent nine tests, the highest pH variations observed were 0.001, 0.03, and 0.002 for tests conducted at pH levels of 7.68, 7.9, and 8, respectively. FIG. 8 illustrates the pH outputs after the serum calibration was established in a Clarke Error Grid. All nine pH values overlap, indicating an acceptable accuracy.

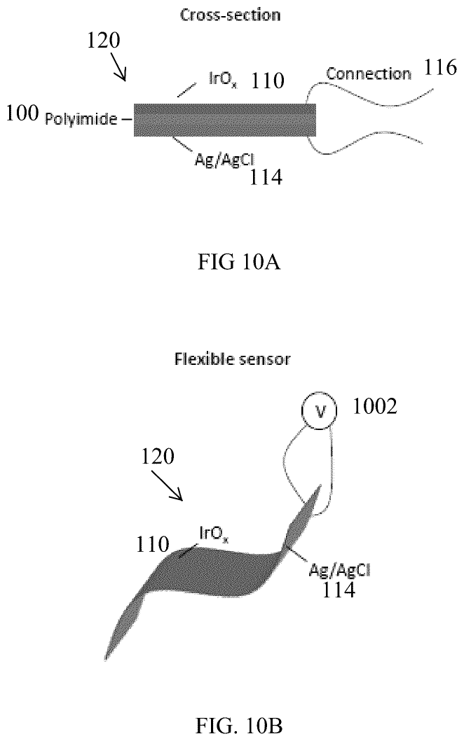

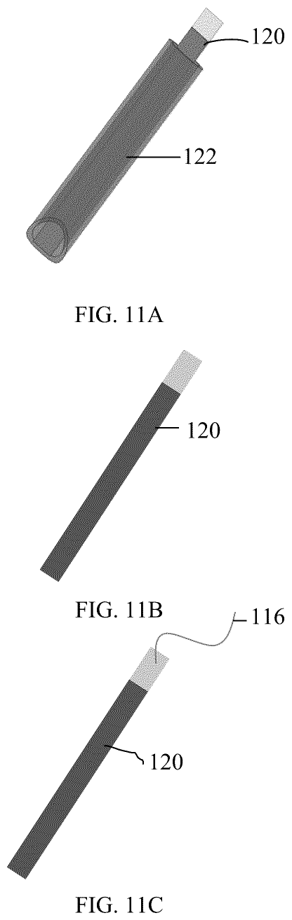

FIG. 9 shows a back-to-back sensing electrode 120 inside a hollow needle 122 that is inserted into tissue including skin 902, blood vessels 904, and fat 906. FIG. 10A shows a cross-section of a back-to-back sensing electrode 120, including a polyimide substrate 100, an iridium oxide working electrode material 110, a silver/silver chloride reference electrode material 114, and a connection to sensing equipment 116. The back-to-back sensing electrode 120 can also use polypropylene, gold bonding wire, platinum wire, or iridium wire for the substrate, among other suitable materials. The working electrode material 110, which is pH-sensitive, can include iridium oxide as shown, or ruthenium oxide, titanium dioxide, tin oxide, tantalum oxide, tungsten oxide, or zinc oxide, among other materials. Iridium oxide is preferred for biocompatibility. The working electrode material 110 can be deposited on the substrate using sol-gel, electroplating, electrodeposition, physical vapor deposition (e.g., E-beam, DC sputtering, and thermal), or metal-organic chemical vapor deposition (MOCVD), among other methods of deposition. The reference electrode material 114 can be silver/silver chloride as shown or mercuric oxide or manganese oxide, among other materials. But mercuric oxide poses a risk of mercury leak in the human body, and manganese oxide can be cytotoxic. Bare silver/silver chloride can also be cytotoxic, but recent studies indicate that silver/silver chloride shows biocompatible properties when coated with a protective layer such as Nafion®. Silver/silver chloride can be deposited on the substrate 100 using commercial silver/silver chloride ink, electroplating or electrodeposition using silver nanoparticles, among other methods of deposition. FIG. 10B shows a perspective view of a back-to-back sensing electrode 120, including an iridium oxide working electrode material layer 110, a silver/silver chloride reference electrode layer 114, and a connection 116 to sensing equipment 1002.

A polypropylene micromembrane can be used as the substrate 100 of a back-to-back sensing electrode 120. Polypropylene and similar fiber materials can be pressed into membrane form. They cannot tolerate high temperatures so the sol-gel method cannot be used to deposit iridium oxide on the surface. A viable method of depositing iridium oxide on such fibers includes depositing gold nanoparticles on the fibers, making the fibers conductive. Then wet electroplating can be used to deposit iridium oxide on the gold nanoparticles. Silver/silver chloride paste can be brush-painted directly onto the fiber substrate. Such fiber substrates are air-permeable so they can be used for wound dressing on tissues, and the fibers are elastic.

FIG. 11A shows a back-to-back sensing electrode 120 inside a needle 122, which can be used to probe into blood vessels. FIG. 11B shows the back-to-back sensing electrode 120 after removal of the needle, leaving the electrode 120 inside the blood vessel. FIG. 11C shows the back-to-back sensing electrode 120 with a connection 116 made to sensing equipment (not shown).

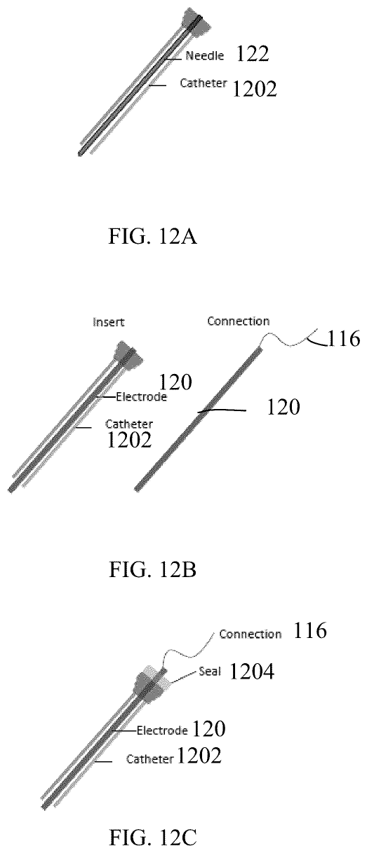

FIG. 12A shows the use of a needle 122 to insert a back-to-back sensing electrode 120 into a blood vessel through an intravenous catheter 1202. FIG. 12B shows the electrode 120 inside the catheter 1202 after removal of the needle 122, and the electrode 120, which remains in the blood vessel, after removal of the catheter 1202. FIG. 12B also shows a connection 116 from the electrode to sensing equipment (not shown). FIG. 12C shows an alternative, leaving the electrode 120 inside the catheter 1202, with a seal 1204 around the electrode 120 where it emerges from the catheter 1204 to be connected via a connection 116 to sensing equipment (not shown). An advantage to the alternative shown in FIG. 12C is that there is no concern about back blood flow and lesser tissue trauma during long-term sensing.

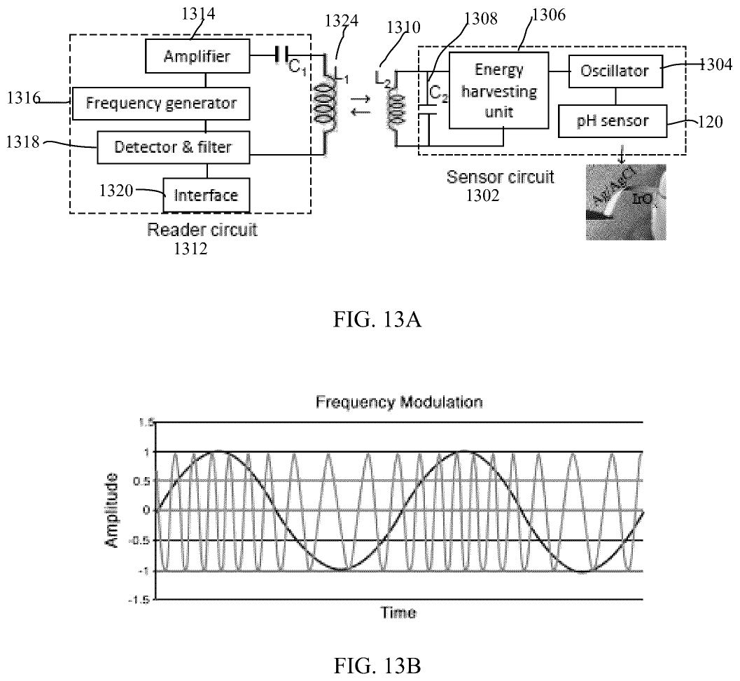

FIG. 13A shows a radio-frequency identification (RFID) implementation of the back-to-back sensing electrode 120 which operates without a battery. The RFID system includes a sensor circuit 1302 and a reader circuit 1312. The sensor circuit 1302 includes a back-to-back sensing electrode 120 that detects pH, an oscillator 1304 that is connected to the electrode 120, an energy harvesting unit 1306 that is connected to the oscillator 1304, and a capacitor C₂ 1308 that is connected to the energy harvesting unit 1306. A coil antenna L₂ 1310 is connected across the capacitor C₂ 1308. The reader circuit 1312 includes an amplifier 1314 that is connected to a frequency generator 1316, a detector and filter 1318 that is connected to the frequency generator 1316, and an interface 1320 that is connected to the detector and filter 1318. The reader circuit 1312 also includes a capacitor C₁ 1322 that is connected to the amplifier 1314 and a coil antenna L₁ 1324, which is connected to the detector and filter 1318 and the amplifier 1314. The sensor circuit 1302 and the reader circuit 1312 are positioned relative to each other such that inductive coupling occurs between the coil antenna L₁ 1324 and the coil antenna L₂ 1310. The back-to-back sensing electrode 120 in operation produces a potential across leads connected to it, and this potential drives the oscillator 1304, a voltage-controlled oscillator that gets a DC bias from the energy harvesting circuit 1306 and produces an AC signal in the kHz range. The AC signal switches the capacitor C₂ 1308, which is tuned to the resonant frequency, 1.3 MHz, between the coil antennas L₁ 1324 and L₂ 1310. When the capacitor C₂ 1308 is switched off, the resonant frequency is detuned, and the kHz-range AC signal from the oscillator 1304 modulates the MHz-range frequency across the gap between the coil antennas L₁ 1324 and L₂ 1310. FIG. 13B shows the signals received at the reader circuit 1312 from the sensor circuit 1302. The filter 1318 of the reader circuit 1312 is a low-pass filter that recovers the AC signal, the frequency of which is related to the voltage generated by the sensor. By counting the AC frequency every one second, the potential output of from the electrode is found.

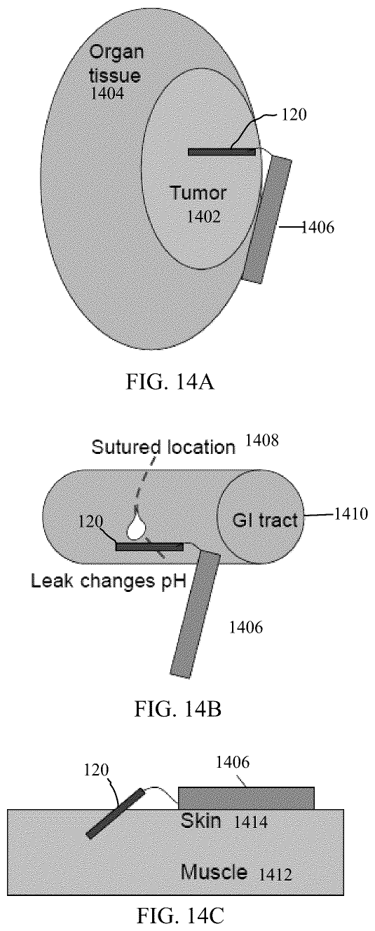

FIG. 14A shows the use of a back-to-back sensing electrode 120 to detect a tumor 1402, with organ tissue 1404 and sensing equipment 1406. FIG. 14B shows the use of the electrode 120 to detect a suture leak 1408 on GI tract 1410, with sensing equipment 1406. FIG. 14C shows the use of the electrode 120 to detect pH in tissue such as muscle tissue 1412, to detect, e.g. lactic acid changes, with skin 1414. In some embodiments, the back-to-back sensing electrode 120 can be connected to sensing equipment 1406 or to a wireless module such as a Bluetooth device (not shown).

In some embodiments, an array of a plurality of back-to-back pH-sensing electrodes can be disposed to form a pH-sensing array to detect pH at a plurality of locations in tissue, such as a wound to assess wound healing or sepsis, or to detect cardiovascular issues, among other applications.

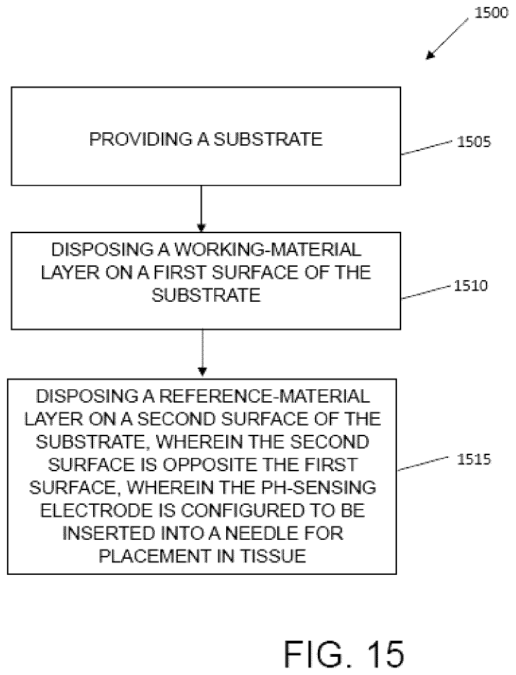

FIG. 15 shows a method embodiment of the present invention. Method 1500 includes block 1505, providing a substrate. Block 1510 includes disposing a working-material layer on a first surface of the substrate. Block 1515 includes disposing a reference-material layer on a second surface of the substrate, wherein the second surface is opposite the first surface, wherein the pH-sensing electrode is configured to be inserted into a needle for placement in tissue.

This study presents a non-limiting example of a miniature pH sensor fabricated back-to-back on a flexible polyimide film and tailored into a strip small enough to fit inside a needle such as a 20-, 28-, or 30-gauge needle. It was designed to detect a small volume of biofluid inside tissues, particularly a blood vessel. The electrode exhibits responsiveness to 0.1 pH variations with repeatability in both buffer solutions and mixed human serum. The ability to detect such subtle pH changes is vital for monitoring human health conditions, given the narrow pH range of human blood (7.35-7.45). The biocompatibility of iridium oxide, coupled with its capability to detect small pH changes, renders it a suitable material for clinical diagnostic purposes. It's simple fabrication process and compact size pave the way for potential integration into medical instruments for early detection of blood infections such as sepsis. The timely identification of sudden pH change is crucial for prompt treatment at the initial stage of infection to avoid catastrophic and potentially deadly outcomes.

Another non-limiting electrode fabrication option that can be used to fabricate all of the electrode or electrode-array configurations disclosed herein includes the following steps. The IrOₓ is deposited and oxidized on a complete piece of the substrate. Then the shape of the electrode shaft is tailored with a sharp blade or other cutting tool such as a laser. The electrode shafts then are placed with the IrOₓ side down and painted or screen printed with Ag/AgCl paste.

Still another non-limiting electrode fabrication option that can be used to fabricate all of the electrode or electrode-array configurations disclosed herein includes the following steps. The substrate is cut to electrode shaft shapes first. Then individual shaft is placed on a carrier wafer with sticky glue to keep them flat. The individual shaft is painted or sprayed with the sol-gel solution. After drying, the individual shafts are removed from the carrier and oxidized with the required temperatures. After cooling down, the other side of shafts are screen-printed or painted with Ag/AgCl and dried.

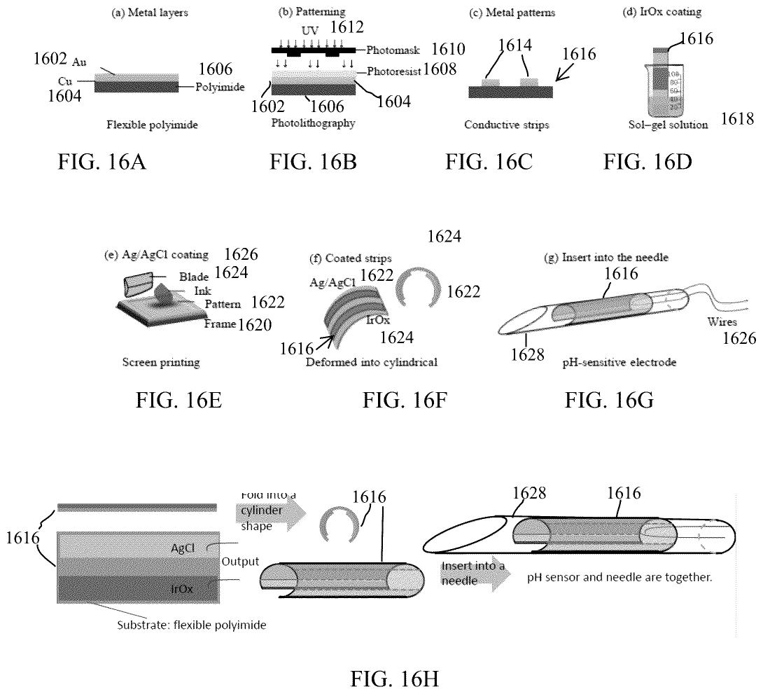

In one specific embodiment of the present invention, a sensing electrode array 1616, a flexible polyimide substrate is coated with copper and gold layers (18 and 90 nm thick, respectively). A layer of photoresist is spin-coated on the substrate and a photomask is applied on the substrate for photolithography to define a pattern of two or more strips separated by a gap. FIG. 16A shows a gold layer 1602, a copper layer 1604, and a flexible substrate 1606. The metals then are etched in gold and copper etchants to form two conductive strips as shown in FIG. 16B, including the gold layer 1602, the copper layer 1604, and the flexible substrate 1606, a photoresist 1608, a photomask 1610, and UV light 1612. FIG. 16C shows two conductive strips 1614 remaining after etching, forming a sensing electrode array 1616. Anhydrous iridium, ethyl alcohol, and 80% acetic acid are mixed as the sol-gel solution 1618. The solution is applied onto one conductive strip 1614 on the substrate as shown in FIG. 16D. This can be done by dipping the sensing electrode array 1616 into the solution 1618 one or multiple times to coat the one conductive strip 1614 with sol-gel solution 1618. It can also be done by dispensing the sol-gel solution 1618 with a nozzle (not shown) onto the one conductive strip 1614. On one conductive strip 1614, the IrOₓ (not shown) is formed by oxidation with the required temperature. After cooling down to the room temperature, the substrate 1606 is placed on a carrier wafer and glued to keep it flat. FIG. 16E shows the Ag/AgCl paste is applied to the other conductive strip on the substrate 1606 using screen printing in which a protection layer (not shown) prevents the paste from being applied on the IrOₓ layer. The screen printing is performed using a frame 1620, a pattern 1622, and ink 1624. A blade 1626 is used to keep the paste thickness uniform. After the paste is dried, the sensing electrode array 1616 is deformed on a cylindrical rod (not shown), with the Ag/AgCl layer 1622 and the IrOₓ layer 1624 facing inward as shown in FIG. 16F. Pressure is applied on the back of the substrate 1606 to ensure a firm contact. The wires 1626 in FIG. 16G are for signal transduction to an external device or circuit. The connection wires 1626 are glued with conductive epoxy. A thin layer of epoxy (not shown) is sprayed on the substrate 1606 back of sensing electrode array 1616. Then the rod (not shown) is inserted into a needle 1628. Once location is confirmed, the sensing electrode array needle 1628 is heated to activate the epoxy allowing the substrate 1606 and the sensing electrode array 1616 as a whole to be glued to the internal wall of the needle 1628. After the glue is dried, the rod (not shown) is withdrawn from the needle 1628. FIG. 16H shows the sensing electrode array 1616 in cross-section, deformed into the shape of a part or whole of a cylinder, and placed in the needle 1628.

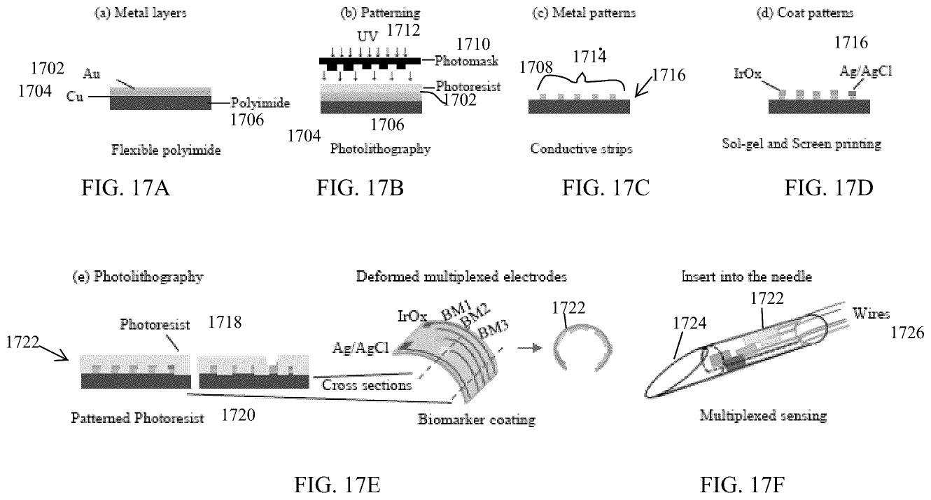

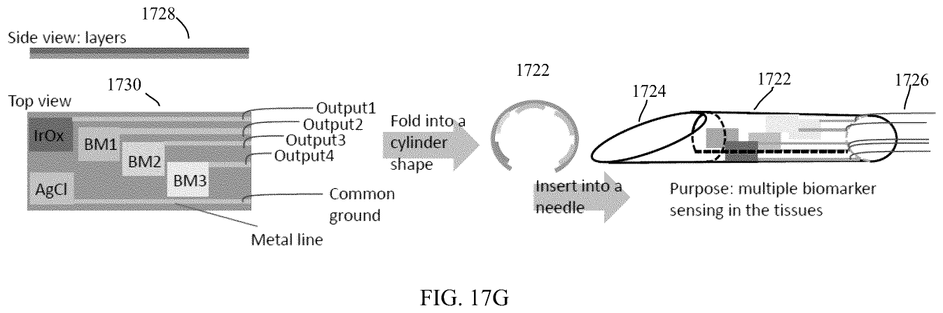

Another specific embodiment of a sensing electrode array 1722 of the present invention includes the following steps. A flexible polyimide substrate is coated with copper and gold layers (18 and 90 nm thick, respectively). A layer of photoresist is spin-coated on the substrate and a photomask is applied on the substrate for photolithography to define electrode patterns and connection lines. FIG. 17A shows a gold layer 1702, a copper layer 1704, and a flexible substrate 1706. The metals then are etched to form patterns on the substrate as shown in FIG. 17B, including the gold layer 1702, the copper layer 1704, and the flexible substrate 1706, a photoresist 1708, a photomask 1710, and UV light 1712. FIG. 17C shows a plurality of conductive strips 1714 remaining after etching, forming a structure 1716. FIG. 17D shows IrOₓ applied to first of the conductive strips 1714. The structure 1716 then goes through the thermal treatment to oxidize and form IrOₓ (not shown). After the substrate is cooled down, Ag/AgCl is applied to a second of the conductive strips 1714 using procedures similar to those used to form sensing electrode array 1616. As shown in FIG. 17E, a layer of photoresist 1718 is applied to structure 1716, and photolithography is applied to expose the conductive strips 1714 while protecting the metal connection lines (not shown). This opens the conductive strips 1714 to liquid but isolates the connection lines (not shown) from being short-circuited. Then the conductive strips 1714 are coated by nozzle-dispensing with target enzymes or receptors 1720 to detect target biochemicals and chemicals to form the sensing electrode array 1722. As shown in FIG. 17E, after the coatings are dried, the sensing electrode array 1722 is rolled on a cylindrical rod and delivered into the needle, similar to the procedures used to deform the pH sensor 1616. After substrate epoxy is dried, the rod is withdrawn leaving the sensing film on the internal wall of the needle. The steps are shown in FIGS. 17E-17F. Specifically, FIGS. 17A-17G show aspects of this embodiment of a sensing electrode array. FIG. 17A shows a metal layer coating on a flexible polyimide substrate. FIG. 17B shows metal patterning by a photolithography process. FIG. 17C shows conductive strips. FIG. 17D shows IrOₓ coating by a sol-gel process. FIG. 17E shows Ag/AgCl coating by screen printing and the sensing electrode array 1722 shaped into a part or a whole of a cylinder. FIG. 17F shows the sensing electrode array 1722 inside a needle 1724 and the wires 1726 for signal transduction. FIG. 17G shows side view 1728 and top view 1730 of the sensing electrode array, a cross-section of the sensing electrode array 1722 shaped into a cylinder, and the sensing electrode array 1722 inside a needle 1724 and the wires 1726 for signal transduction. FIG. 17G's top view 1730 of the sensing electrode array shows the working electrode IrOₓ for pH sensing, while the working electrodes BM1, BM2 and BM3 detect biomarkers 1, 2, and 3, respectively. All working electrodes use AgCl electrode as the reference electrode. Outputs #1-4 are the potentials between the respective working electrodes and reference electrode.

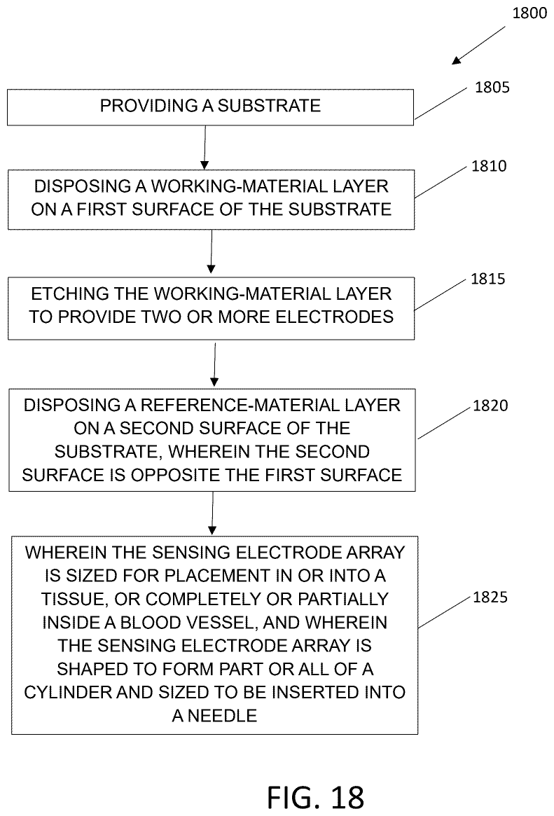

FIG. 18 shows a method 1800 of the present invention. Method 1800 includes block 1805, providing a substrate. Method 1800 also includes block 1810, disposing a working-material layer on a first surface of the substrate; and block 1815, etching the working-material layer to provide two or more electrodes. Further, Method 1800 includes block 1820, disposing a reference-material layer on a second surface of the substrate, wherein the second surface is opposite the first surface. In addition, method 1800 includes block 1825, wherein the sensing electrode array is sized for placement in or into a tissue, or completely or partially inside a blood vessel and wherein the sensing electrode array is shaped to form part or all of a cylinder and sized to be inserted into a needle.

The sensing electrodes and sensing electrode arrays of the present invention can be configured to detect various biomarkers to detect medical conditions, e.g., a level of glucose to detect hypoglycemia or hyperglycemia, a level of lactic acid to monitor tissue oxygenation, identify lactic acidosis and diagnose and monitor sepsis, shock and heart failure, levels of electrolytes such as sodium, potassium and calcium during and after surgery to prevent imbalances, antibody and antigen binding for infection diagnosis, and other biochemicals for acute and chronic disorders.

The sensing electrodes and sensing electrode arrays of the present invention can be configured to be wearable by a patient. The sensing electrodes and sensing electrode arrays of the present invention can also be configured to operate wirelessly. The surfaces or surfaces of the working-material layers of the electrodes and sensing electrode arrays of the present invention that are exposed to the tissue being probed can also be roughened to increase the surface area, which increases sensitivity. The sensing electrodes and sensing electrode arrays of the present invention can be configured to be placed in an artery, a vein, GI tract, or configured to be placed in one or more of these types of insertion: subcutaneous, subdermal, intraperitoneal, intramuscular, intraarticular, periarticular, intraosseous, intraocular, intracardiac, intracavernous, intradetrusor, or other types of insertion that are useful for pH monitoring.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of.” As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step, or a limitation) or group of integers (e.g., feature(s), element(s), characteristic(s), property (ies), method/process(s) steps, or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the devices and/or methods disclosed and claimed herein can be made and executed without undue experimentation in light of the present disclosure. While the devices and/or methods of this invention have been described in terms of particular embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope, and concept of the invention as defined by the appended claims.

Furthermore, no limitations are intended to the details of construction or design herein shown, other than as described in the claims below. It is therefore evident that the particular embodiments disclosed above may be altered or modified and all such variations are considered within the scope and spirit of the disclosure. Accordingly, the protection sought herein is as set forth in the claims below.

Modifications, additions, or omissions may be made to the systems and apparatuses described herein without departing from the scope of the invention. The components of the systems and apparatuses may be integrated or separated. Moreover, the operations of the systems and apparatuses may be performed by more, fewer, or other components. The methods may include more, fewer, or other steps. Additionally, steps may be performed in any suitable order.

To aid the Patent Office, and any readers of any patent issued on this application in interpreting the claims appended hereto, applicants wish to note that they do not intend any of the appended claims to invoke 35 U.S.C. § 112 (f) as it exists on the date of filing hereof unless the words “means for” or “step for” are explicitly used in the particular claim.

### REFERENCES

[1] S. Lim, “Metabolic acidosis,” Acta Med Indones, vol. 39, pp. 145-150, 2007.
[2] X. Zhang, Y. Lin and R. J. Gillies, “Tumor pH and its measurement,” J. of Nuclear Med., vol. 51, pp. 1167-1170, 2010.
[3] A. U. Alam et al., “Polymers and organic materials-based pH sensors for healthcare applications,” Progress in Material Science, vol. 96, pp. 174-216, 2018.
[4] H. Lambers et al., “Nat. skin surf. pH is on ave. below 5, which is benef. for its resident flora,” Int. J. Cosmetic Sci., vol. 28, pp. 359-370, 2006.
[5] T. J. Durkin, B. Barua and S. Savagatrup, “Rapid detection of sepsis: Recent advances in biomarker sensing platforms,” ACS Omega, vol. 6, pp. 31390-31395, 2021.
[6] A. P. Murphy and P. Rolfe, “Assessment of a commercial pH electrode used with blood,” J. Med. Eng. Technol., vol. 15, pp. 116-118, 1991.
[7] R. N. Khuri, S. K. Agulian and R. I. Harik, “The liquid junction potential in blood pH determinations,” Physics in Medicine & Biology, vol. 13, pp. 23, 1968.
[8] D. J. Alner, J. J. Greczek and A. G. Smeeth, “Precise standard pH values from 0 to 60°,” J. Chem. Soc. A, pp. 1205-1211, 1967.
[9] D. Choi et al., “A wearable poten. sensor with integrated salt bridge for sweat chloride meas.,” Sens Actuators B Chem., vol. 250, pp. 673-678, 2017.
[10] H. Y. Y. Nyein et al. “A wearable electro. platform for noninvasive simultaneous monitoring of Ca2 and pH,” ACS Nano, vol. 10, pp. 7216-7224, 2016.
[11] R. Wang et al., “Stretchable gold fiber-based wearable electrochem. sensor toward pH monitoring,” J. of Mat. Chem. B, vol. 8, pp. 3655-3660, 2020.
[12] P. Chao, D. Ammann, U. Oesch, W. Simon and F. Lang, “Extra- and intra. hydrogen ion-selective microelectrode based on neutral carriers with extended pH response range in acid media,” Pflügers Archiv, vol. 411, pp. 216-219, 1988.
[13] Y. Zhang et al., “Synthesis and biocompatibility assessment of PANI nanomaterials,” J. Bioact. Compatible Polym., vol. 34, pp. 16-24, 2019.
[14] Y. Tang et al., “Recent advances in wearable potentiometric pH sensors,” Membranes, vol. 12, pp. 504, 2022.
[15] D. O'Hare, K. H. Parker and C. P. Winlove, “Metal-metal oxide pH sensors for physiological application,” Med. Eng. Phys., vol. 28, pp. 982-988, 2006.
[16] R. A. Frederick, I. Y. Meliane, A. Joshi-Imre, P. R. Troyk and S. F. Cogan, “AIROF electrodes for neural tissue stimul.,” J. of Neural Eng., vol. 17, pp. 056001, 2020.
[17] K. Chaisiwamongkhol, C. Batchelor and R. G. Compton, “pH sensing blood samples Iridium for blood pH sensing,” Analyst, vol. 144, pp. 1386-1393, 2019.
[18] J. Yang et al., “Digital pH test strips using iridium oxide hybrid thin films,” Acs Sensors, vol. 1, pp. 1235-1243, 2016.
[19] K. Göbbels et al., “Neuronal cell growth on iridium oxide,” Biomaterials, vol. 31, pp. 1055-1067, 2010.
[20] K. Izutsu and H. Yamamoto, “Iridium Oxide pH-Sensor in Nonaqueous Solution with Other pH-Sensors,” Anal. Sci., vol. 12, pp. 905-909, Dec. 10, 1996.
[21] X. Huang, Q. Ren, X. Yuan, W. Wen, W. Chen and D. Zhan, “Iridium oxide based coaxial pH ultramicroelectrode,” Electrochem. Commm., vol. 40, pp. 35-37, 2014.
[22] M. E. Khakani, M. Chaker and B. L. Drogoff, “Iridium thin films deposited by RF magnetron sputtering,” J. Vac. Sci. and Tech. A, vol. 16, pp. 885-888, 1998.
[23] E. Slavcheva, R. Vitushinsky, W. Mokwa and U. Schnakenberg, “SIROF as charge injection mat. for functional electrosti.,” J. Elec. Soc., vol. 151, pp. E226, 2004.
[24] R. Y. Bekele et al., “CIGS sputtering targets fabricated from reclaimed materials,” in 2013 IEEE 39th PVSC, pp. 1938-1940, 2013.
[25] P. Savignac et al., “Improvement of adhesion properties and corrosion resistance of sol-gel coating on zinc,” Molecules, vol. 23, pp. 1079, 2018.
[26] W. S. Al-Arjan et al., “Sol-gel preparation of well-adhered films of BaTiO3 and Bi2Ti2O7,” Mater. Res. Bull., vol. 74, pp. 234-240, 2016.
[27] X. Yang and J.-C. Chiao, “Integrated pH and Sodium Sensor Array Based on Iridium Oxide Film,” in IEEE Sensors Conference, India, Oct. 28-31, 2018.
[28] W. Huang et al., “A Passive RF pH-Sensing Tag for Wireless Food-Quality Monitoring,” IEEE Sensors Journal, vol. 12, pp. 487-495, 2011.
[29] K. Chawang, S. Bing and J.-C. Chiao, “Printable and Flexible Iridium Oxide-Based pH Sensor by a Roll-to-Roll Process,” Chemosensors, vol. 11, pp. 267, 2023.
[30] H. R. Pohl, J. S. Wheeler and H. E. Murray, “Sod. and pot. in health and disease,” Interr. bet. Essential Metal Ions and Human Diseases, pp. 29-47, 2013.
[31] Y. Liao and J. Chou, “Titanium dioxide thin films used for pH electrode and procaine drug sensor by sol-gel method,” Mater. Chem. Phys., vol. 114, pp. 542-548, 2009.
[32] D. H. Kim et al., “20-gauge ProCore needle in EUS-guided subepithelial tumor sampling: a prospective multicenter study,” BMC Gas., vol. 18, pp. 1-7, 2018.
[33] G. Geleto, W. Getnet and T. Tewelde, “Mean vein diameter using sonography, Southwest Ethiopia,” Ethiopian J. of Health Sci., vol. 26, pp. 237-242, 2016.

What is claimed is:

1. A pH-sensing electrode comprising:
a substrate;
a working-material layer disposed on a first surface of the substrate; and
a reference-material layer disposed on a second surface of the substrate, wherein the second surface is opposite the first surface;
wherein the pH-sensing electrode is sized for placement in or into a tissue, or completely or partially inside a blood vessel.

2. The pH-sensing electrode of claim 1, wherein the substrate comprises at least one of: polyimide, ethylene-tetrafluoroethylene (ETFE), a fluorinated polymer, polyester, polyamide, polyvinylidene fluoride (PVDF), polycarbonate, ETFE/PVDF mixtures, polypropylene, aromatic polyethers, polybenzoxazoles, polyetheretherketones, polybenzimidazoles, glass, epoxy resin, bismaleimide, cyanate ester, vinyl resin, polyethersulfone, polyurethane, gold bonding wire, platinum wire, iridium wire, or combinations thereof.

3. The pH-sensing electrode of claim 1, wherein the working-material layer comprises at least one of: iridium oxide, ruthenium oxide, titanium dioxide, tin oxide, tantalum oxide, tungsten oxide, erbium oxide, cerium oxide, or zinc oxide.

4. The pH-sensing electrode of claim 1, wherein the working-material layer is deposited on the substrate by at least one of: a sol-gel method, electroplating, electrodeposition, physical vapor deposition, or metal-organic chemical vapor deposition.

5. The pH-sensing electrode of claim 1, wherein the reference-material layer comprises at least one of: silver/silver chloride, mercuric oxide, or manganese oxide.

6. The pH-sensing electrode of claim 1, wherein the reference-material layer is deposited on the substrate by at least one of: silver/silver chloride ink deposition, electroplating using silver nanoparticles, or electrodeposition using silver nanoparticles.

7. The pH-sensing electrode of claim 1, wherein the pH-sensing electrode is inserted into a needle.

8. The pH-sensing electrode of claim 1, wherein the pH-sensing electrode is adapted for subcutaneous, percutaneous, intracutaneous, subdermal, intraperitoneal, intramuscular, intraarticular, periarticular, intraosseous, intraocular, intracardiac, intracavernous, intradetrusor insertion, or insertion into a vein or artery.

9. A pH-sensing array comprising:
a plurality of pH-sensing electrodes, each pH-sensing electrode comprising:
a substrate;
a working-material layer disposed on a first surface of the substrate; and
a reference-material layer disposed on a second surface to the substrate, wherein the second surface is opposite the first surface;
wherein the pH-sensing electrode is configured for placement in or into a tissue, or completely or partially inside a blood vessel.

10. The pH-sensing array of claim 9, wherein the substrate comprises at least one of: polyimide, ethylene-tetrafluoroethylene (ETFE), a fluorinated polymer, polyester, polyamide, polyvinylidene fluoride (PVDF), polycarbonate, ETFE/PVDF mixtures, polypropylene, aromatic polyethers, polybenzoxazoles, polyetheretherketones, polybenzimidazoles, glass, epoxy resin, bismaleimide, cyanate ester, vinyl resin, polyethersulfone, polyurethane, gold bonding wire, platinum wire, iridium wire, or combinations thereof.

11. The pH-sensing array of claim 9, wherein the working-material layer comprises at least one of: iridium oxide, ruthenium oxide, titanium dioxide, tin oxide, tantalum oxide, tungsten oxide, erbium oxide, cerium oxide, or zinc oxide.

12. The pH-sensing array of claim 9, wherein the working-material layer is deposited on the substrate by at least one of: a sol-gel method, electroplating, electrodeposition, physical vapor deposition, or metal-organic chemical vapor deposition.

13. The pH-sensing array of claim 9, wherein the reference-material layer comprises at least one of: silver/silver chloride, mercuric oxide, or manganese oxide.

14. The pH-sensing array of claim 9, wherein the reference-material layer is deposited on the substrate by at least one of: silver/silver chloride ink deposition, electroplating using silver nanoparticles, or electrodeposition using silver nanoparticles.

15. The pH-sensing array of claim 9, wherein the plurality of pH-sensing electrodes are adapted for subcutaneous, percutaneous, intracutaneous, subdermal, intraperitoneal, intramuscular, intraarticular, periarticular, intraosseous, intraocular, intracardiac, intracavernous, intradetrusor insertion, or insertion into a vein or artery.

16. A method of making one or more pH-sensing electrodes comprising:
providing a substrate;
disposing a working-material layer on a first surface of the substrate; and
disposing a reference-material layer on a second surface of the substrate, wherein the second surface is opposite the first surface;
wherein the pH-sensing electrode is sized for placement in or into a tissue, or completely or partially inside a blood vessel.

17. The method of claim 16, wherein the substrate comprises at least one of: polyimide, ethylene-tetrafluoroethylene (ETFE), a fluorinated polymer, polyester, polyamide, polyvinylidene fluoride (PVDF), polycarbonate, ETFE/PVDF mixtures, polypropylene, aromatic polyethers, polybenzoxazoles, polyetheretherketones, polybenzimidazoles, glass, epoxy resin, bismaleimide, cyanate ester, vinyl resin, polyethersulfone, polyurethane, gold bonding wire, platinum wire, iridium wire, or combinations thereof.

18. The method of claim 16, wherein the working-material layer comprises at least one of: iridium oxide, ruthenium oxide, titanium dioxide, tin oxide, tantalum oxide, tungsten oxide, erbium oxide, cerium oxide, or zinc oxide.

19. The method of claim 16, wherein the working-material layer is deposited on the substrate by at least one of: a sol-gel method, electroplating, electrodeposition, physical vapor deposition, or metal-organic chemical vapor deposition.

20. The method of claim 16, wherein the reference-material layer comprises at least one of: silver/silver chloride, mercuric oxide, or manganese oxide.

21. The method of claim 16, wherein the reference-material layer is deposited on the substrate by at least one of: silver/silver chloride ink deposition, electroplating using silver nanoparticles, or electrodeposition using silver nanoparticles.

22. The method of claim 16, wherein the pH-sensing electrode is adapted for insertion into a needle.

23. The method of claim 16, wherein the one or more pH-sensing electrodes are adapted for subcutaneous, percutaneous, intracutaneous, subdermal, intraperitoneal, intramuscular, intraarticular, periarticular, intraosseous, intraocular, intracardiac, intracavernous, intradetrusor insertion, or insertion into a vein or artery.

24. A sensing electrode array comprising:
a substrate;
a working-material layer disposed on a first surface of the substrate and etched to provide two or more electrodes; and
a reference-material layer disposed on a second surface of the substrate, wherein the second surface is opposite the first surface;
wherein the sensing electrode is sized for placement in or into a tissue, or completely or partially inside a blood vessel; and
wherein the sensing electrode is shaped to form part or all of a cylinder and sized to be inserted into a needle.

25. The sensing electrode array of claim 24, wherein the substrate comprises at least one of: polyimide, ethylene-tetrafluoroethylene (ETFE), a fluorinated polymer, polyester, polyamide, polyvinylidene fluoride (PVDF), polycarbonate, ETFE/PVDF mixtures, polypropylene, aromatic polyethers, polybenzoxazoles, polyetheretherketones, polybenzimidazoles, glass, epoxy resin, bismaleimide, cyanate ester, vinyl resin, polyethersulfone, polyurethane, gold bonding wire, platinum wire, iridium wire, or combinations thereof.

26. The sensing electrode array of claim 24, wherein the working-material layer comprises at least one of: iridium oxide, ruthenium oxide, titanium dioxide, tin oxide, tantalum oxide, tungsten oxide, erbium oxide, cerium oxide, or zinc oxide.

27. The sensing electrode array of claim 24, wherein the working-material layer is deposited on the substrate by at least one of: a sol-gel method, electroplating, electrodeposition, physical vapor deposition, or metal-organic chemical vapor deposition.

28. The sensing electrode array of claim 24, wherein the reference-material layer comprises at least one of: silver/silver chloride, mercuric oxide, or manganese oxide.

29. The sensing electrode array of claim 24, wherein the reference-material layer is deposited on the substrate by at least one of: silver/silver chloride ink deposition, electroplating using silver nanoparticles, or electrodeposition using silver nanoparticles.

30. The sensing electrode array of claim 24, wherein the one or more pH-sensing electrodes are adapted for subcutaneous, percutaneous, intracutaneous, subdermal, intraperitoneal, intramuscular, intraarticular, periarticular, intraosseous, intraocular, intracardiac, intracavernous, intradetrusor insertion, or insertion into a vein or artery.

31. A method of making a sensing electrode array comprising:
providing a substrate;
disposing a working-material layer on a first surface of the substrate;
etching the working-material layer to provide two or more electrodes; and
disposing a reference-material layer on a second surface of the substrate, wherein the second surface is opposite the first surface;
wherein the sensing electrode array is sized for placement in or into a tissue, or completely or partially inside a blood vessel, and wherein the sensing electrode array is shaped to form part or all of a cylinder and sized to be inserted into a needle.

32. The method of claim 31, wherein the substrate comprises at least one of: polyimide, ethylene-tetrafluoroethylene (ETFE), a fluorinated polymer, polyester, polyamide, polyvinylidene fluoride (PVDF), polycarbonate, ETFE/PVDF mixtures, polypropylene, aromatic polyethers, polybenzoxazoles, polyetheretherketones, polybenzimidazoles, glass, epoxy resin, bismaleimide, cyanate ester, vinyl resin, polyethersulfone, polyurethane, gold bonding wire, platinum wire, iridium wire, or combinations thereof.

33. The method of claim 31, wherein the working-material layer comprises at least one of: iridium oxide, ruthenium oxide, titanium dioxide, tin oxide, tantalum oxide, tungsten oxide, or zinc oxide.

34. The method of claim 31, wherein the working-material layer is deposited on the substrate by at least one of: a sol-gel method, electroplating, electrodeposition, physical vapor deposition, or metal-organic chemical vapor deposition.

35. The method of claim 31, wherein the reference-material layer comprises at least one of: silver/silver chloride, mercuric oxide, or manganese oxide.

36. The method of claim 31, wherein the reference-material layer is deposited on the substrate by at least one of: silver/silver chloride ink deposition, electroplating using silver nanoparticles, or electrodeposition using silver nanoparticles.

37. The method of claim 31, wherein the two or more pH-sensing electrodes are adapted for insertion into a needle.

38. The method of claim 31, wherein the two or more pH-sensing electrodes are adapted for subcutaneous, percutaneous, intracutaneous, subdermal, intraperitoneal, intramuscular, intraarticular, periarticular, intraosseous, intraocular, intracardiac, intracavernous, intradetrusor insertion, or insertion into a vein or artery.
